(19) United States
(12) Patent Application Publication
(10) Pub. No.: US 2006/0271033 A1
(43) Pub. Date: Nov. 30, 2006

(54) Tissue ablation with jet injection of conductive fluid

(76) Inventor: Moxhe Ein-Gal, Ramat Hasharon (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/140,204

(22) Filed: May 31, 2005

(51) Int. Cl. A61B 18/14

(52) U.S. Cl. 606/41

(57) ABSTRACT

Ablation apparatus including an active electrode in electrical communication with an energy source, and a jet injector capable of propelling an electrically conducting fluid to a skin surface, the jet injector being positioned relative to the active electrode such that the electrically conducting fluid is in electrical communication with the active electrode.

### FIELD OF THE INVENTION

The present invention relates generally to devices for tissue ablation, and particularly to such a device that employs a jet injection of conductive fluid for electrode ablation.

### BACKGROUND OF THE INVENTION

The field of electrosurgery includes a number of loosely related surgical techniques which have in common the application of electrical energy to modify the structure or integrity of patient tissue. Electrosurgical procedures usually operate through the application of very high frequency currents to cut or ablate tissue structures, where the operation can be monopolar or bipolar. Monopolar techniques rely on external grounding of the patient, where the surgical device defines only a single electrode pole. Bipolar devices comprise both electrodes for the application of current between their surfaces.

Electrosurgical procedures and techniques are particularly advantageous since they generally reduce patient bleeding and trauma associated with cutting operations. Current electrosurgical device and procedures, however, suffer from a number of disadvantages. For example, monopolar devices generally direct electric current along a defined path from the exposed or active electrode through the patient's body to the return electrode, which is externally attached to a suitable location on the patient. This creates a potential danger that the electric current will flow through undefined paths in the patient's body, thereby increasing the risk of unwanted electrical stimulation to portions of the patient's body. In addition, since the defined path through the patient's body has a relatively high impedance (because of the large distance or resistivity of the patient's body), large voltage differences must typically be applied between the return and active electrodes in order to generate a current suitable for ablation or cutting of the target tissue. This current, however, may inadvertently flow along body paths having less impedance than the defined electrical path, which will substantially increase the current flowing through these paths, possibly causing damage to or destroying tissue along and surrounding this pathway.

Bipolar electrosurgical devices have an inherent advantage over monopolar devices because the return current path does not flow through the patient. In bipolar electrosurgical devices, both the active and return electrodes are typically exposed so that they may both contact tissue, thereby providing a return current path from the active to the return electrode through the tissue. One drawback with this configuration, however, is that the return electrode may cause tissue desiccation or destruction at its contact point with the patient's tissue. In addition, the active and return electrodes are typically positioned close together to ensure that the return current flows directly from the active to the return electrode. The close proximity of these electrodes generates the danger that the current will short across the electrodes, possibly impairing the electrical control system and/or damaging or destroying surrounding tissue.

U.S. Pat. No. 6,773,431 describes a system and method for selectively applying electrical energy to structures within or on the surface of a patient's body. The method includes positioning an electrosurgical probe adjacent the target tissue so that at least one active electrode is brought into close proximity to the target site. A return electrode is positioned within an electrically conducting liquid, such as isotonic saline, to generate a current flow path between the target site and the return electrode. High frequency voltage is then applied between the active and return electrode through the current flow path created by the electrically conducting liquid in either a bipolar or monopolar manner. The probe may then be translated, reciprocated or otherwise manipulated to cut the tissue or effect the desired depth of ablation. The current flow path is generated by directing an electrically conducting liquid along a fluid path past the return electrode and to the target site to generate the current flow path between the target site and the return electrode.

U.S. Pat. No. 6,241,753 describes a method for epidermal ablation at a selected collagen containing tissue site. The method includes producing energy from an energy source, creating a reverse thermal gradient through the skin epidermis surface where a temperature of the skin epidermis surface is lower than the selected collagen containing tissue site, and delivering energy from the energy source through the skin epidermis surface to the selected collagen containing tissue site for a sufficient time to induce collagen formation in the selected collagen containing tissue site, minimizing cellular necrosis of the skin epidermis surface and tightening the skin epidermis surface.

U.S. Pat. No. 6,149,620 describes systems and methods for electrosurgical tissue treatment in the presence of electrically conductive fluid. This involves applying a high frequency voltage in the presence of an electrically conductive fluid to create a relatively low-temperature plasma for ablation of tissue adjacent to, or in contact with, the plasma. In one embodiment, an electrosurgical probe or catheter is positioned adjacent the target site so that one or more active electrode(s) are brought into contact with, or close proximity to, a target tissue in the presence of electrically conductive fluid. High frequency voltage is then applied between the electrode terminal(s) and one or more return electrode(s) to generate a plasma adjacent to the active electrode(s), and to volumetrically remove or ablate at least a portion of the target tissue.

U.S. Pat. No. 6,702,810 describes a system for treating tissue that includes a power measurement device, a flow rate controller coupled to the power measurement device, and an electrosurgical device configured and arranged to provide radio frequency power and conductive fluid to the tissue, wherein the flow rate controller is configured and arranged to modify a flow rate of the conductive fluid to the tissue, based on signals from the power measurement device.

### SUMMARY OF THE INVENTION

The present invention seeks to provide novel tissue ablation apparatus, which employs a jet injection of conductive fluid for electrode ablation (e.g., monopolar or bipolar), as is described more in detail hereinbelow.

The present invention may be used for the treatment of tissue underneath the epidermis without damaging the skin. In a non-limiting embodiment of the present invention, a perforated non-contact RF electrode may be placed close to the patient's skin. The active electrode is operable to form jets of conducting liquid passing through one or more electrode perforations. The jets impinge and may pierce the skin. The conducting jets, being in contact with the perforated electrode, carry electricity to the skin and through the skin to the underlying tissue (the circuit may be closed by a return electrode). The liquid may be collected, cooled and recirculated.

There is thus provided in accordance with an embodiment of the present invention ablation apparatus including an active electrode in electrical communication with an energy source, and a jet injector capable of propelling an electrically conducting fluid to pierce through a skin surface, the jet injector being positioned relative to the active electrode such that the electrically conducting fluid is in electrical communication with the active electrode.

The active electrode may be formed with fluid passageways in fluid communication with the jet injector, wherein the electrically conducting fluid flows from the jet injector through the fluid passageways.

A reservoir of electrically conducting fluid may be in fluid communication with the jet injector. Fluid collection apparatus may be adapted to collect electrically conducting fluid discharged from the jet injector and to return the electrically conducting fluid to the reservoir.

### BRIEF DESCRIPTION OF THE DRAWINGS

The present invention will be understood and appreciated more fully from the following detailed description taken in conjunction with the appended drawing in which:

FIG. 1 is a simplified schematic illustration of ablation apparatus, constructed and operative in accordance with an embodiment of the present invention.

### DETAILED DESCRIPTION OF THE PRESENT INVENTION

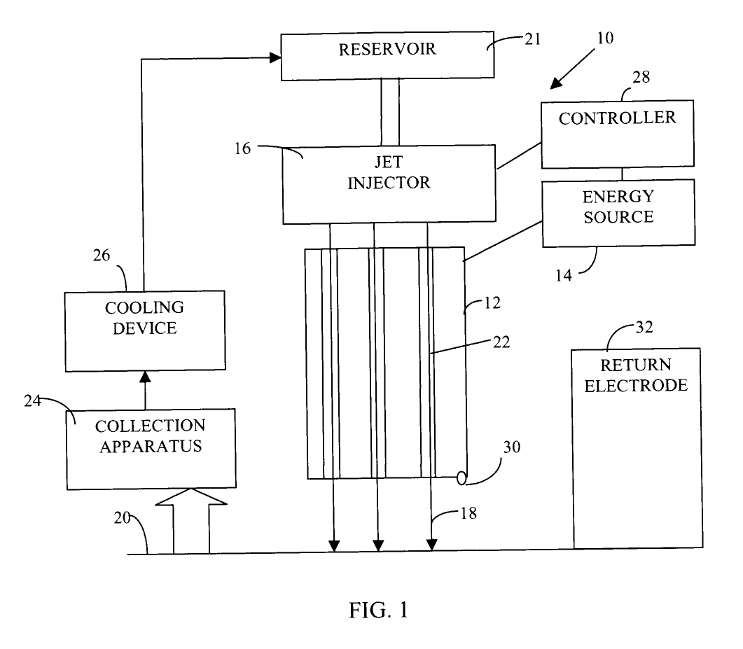

Reference is now made to FIG. 1, which illustrates ablation apparatus 10, constructed and operative in accordance with an embodiment of the present invention.

Ablation apparatus 10 may include an active electrode 12 in electrical communication with an energy source 14. Energy source 14 may be associated with an intense electric field, energetic photons or energetic electrons, for example. Typically, energy source 14 may be an RF (radio frequency) or high frequency voltage (typically between about 5 kHz and 20 MHz, but not limited to this range).

A jet injector 16 is provided, which is capable of propelling an electrically conducting fluid 18 to a target site, such as a skin surface, 20. Jet injector 16 may propel the fluid 18 to pierce through the skin surface. Alternatively, the invention may be carried out without piercing the skin surface. A reservoir 21 of electrically conducting fluid 18 may be in fluid communication with jet injector 16. The jet injector 16 is positioned relative to the active electrode 12 such that the electrically conducting fluid 18 is in electrical communication with the active electrode 12. For example, the active electrode 12 may be formed with fluid passageways 22 in fluid communication with the jet injector 16, wherein the electrically conducting fluid 18 flows from the jet injector 16 through the fluid passageways 22 towards the target site 20.

As is well known in the art, a needle-less jet injector typically may use either a mechanical system (e.g., compression spring) or a pneumatic/hydraulic system (e.g., compressed inert gas) to propel fluid (e.g., fluid medication) through a small orifice (an injector nozzle) which is generally perpendicular to the injection site. The propulsion accelerates a fine stream of fluid at relatively high velocity (such as but not limited to, approximately 200-400 meters per second) and pressure so that the fluid penetrates the skin and deposits subcutaneously in the tissue. Accordingly, the jet injector 16 is capable of propelling the electrically conducting fluid 18 to deliver ablative energy from the active electrode 12 to a site under the skin surface.

The electrically conducting fluid 18 may comprise a saline solution, for example. The electrically conducting fluid 18 may include a medicinal substance, such as but not limited to, a medication or analgesic drug.

In accordance with a non-limiting embodiment of the present invention, fluid collection apparatus 24 may be provided, which can collect electrically conducting fluid 18 discharged from the jet injector 16 and can return the electrically conducting fluid 18 to the reservoir 21. For example, fluid collection apparatus 24 may include a suction or aspiration device to suck the electrically conducting fluid 18 and to pump the fluid 18 back to reservoir 21. A cooling device 26 may be provided for cooling the electrically conducting fluid 18 discharged from the jet injector 16 (downstream or upstream or independent of fluid collection apparatus 24). The cooling device 26 may include a liquid-to-air heat exchanger or liquid-to-liquid heat exchanger, for example.

A controller 28 may be operatively connected to the jet injector 12 and the energy source 14. Controller 28 may control operation of jet injector 12 and energy source 14. For example, controller 28 may operate in a close loop control with a temperature sensor 30 located in a vicinity of the active electrode 12 to control the energy from energy source 14 and the jet action of jet injector 16, thereby to control ablation speed, depth and other parameters.

The active electrode 12 may operate in a monopolar mode of operation, relying on external grounding of the patient. Alternatively, a return electrode 32 may be provided for operating in a bipolar mode of operation with the active electrode 12, wherein the return electrode 32 is in electrical communication with the active electrode 12 via the electrically conducting fluid 18. The return electrode 32 may be spaced from the active electrode 12 and enclosed within an insulating sheath, for example. Alternatively return electrode 32 may be placed at other locations.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Ablation apparatus comprising:
an active electrode in electrical communication with an energy source; and
a jet injector capable of propelling an electrically conducting fluid to a skin surface, said jet injector being positioned relative to said active electrode such that said electrically conducting fluid is in electrical communication with said active electrode.

2. The ablation apparatus according to claim 1, wherein said jet injector is capable of propelling the electrically conducting fluid to deliver ablative energy from said active electrode to a site under said skin surface.

3. The ablation apparatus according to claim 1, wherein said active electrode is formed with fluid passageways in fluid communication with said jet injector, wherein said electrically conducting fluid flows from said jet injector through said fluid passageways.

4. The ablation apparatus according to claim 1, further comprising a reservoir of electrically conducting fluid in fluid communication with said jet injector.

5. The ablation apparatus according to claim 4, further comprising fluid collection apparatus adapted to collect electrically conducting fluid discharged from said jet injector and to return said electrically conducting fluid to said reservoir.

6. The ablation apparatus according to claim 1, further comprising a cooling device for cooling said electrically conducting fluid discharged from said jet injector.

7. The ablation apparatus according to claim 1, further comprising a controller operatively connected to and capable of controlling operation of said jet injector and said energy source.

8. The ablation apparatus according to claim 1, further comprising a temperature sensor in a vicinity of said active electrode.

9. The ablation apparatus according to claim 1, further comprising a return electrode adapted to operate in a bipolar mode of operation with said active electrode, said return electrode being in electrical communication with said active electrode via said electrically conducting fluid.

10. The ablation apparatus according to claim 4, wherein said electrically conducting fluid comprises a medicinal substance.

11. Ablation apparatus comprising:
an energy source;
an active electrode in electrical communication with said energy source; and
an electrically conducting fluid in electrical communication with said active electrode, wherein said electrically conducting fluid comprises a medicinal substance.

12. The ablation apparatus according to claim 11, further comprising a jet injector capable of propelling said electrically conducting fluid to a skin surface.
